(19) United States
(12) Patent Application Publication
(10) Pub. No.: US 2022/0387009 A1
(43) Pub. Date: Dec. 8, 2022

(54) SYSTEMS AND METHODS FOR TEMPORARY SHUNTING BETWEEN HEART CHAMBERS

(71) Applicant: V-Wave Ltd., Caesarea (IL)

(72) Inventors: Nathan BUKHDRUKER, Haifa (IL); Nir NAE, Binyamina (IL); Lior ROSEN, Zikhron Ya'akov (IL); Erez ROZENFELD, Shoham (IL); James S. WHITING, Los Angeles, CA (US); Werner HAFELFINGER, Thousand Oaks, CA (US); Neal EIGLER, Angoura Hills, CA (US); Gad KEREN, Kiryat-Ono (IL)

(73) Assignee: V-Wave Ltd., Caesarea (IL)

(21) Appl. No.: 17/805,001

(22) Filed: Jun. 1, 2022

Related U.S. Application Data
(60) Provisional application No. 63/197,279, filed on Jun. 4, 2021.

(51) Int. Cl. A61B 17/00

(52) U.S. Cl. CPC A61B 17/0057; A61B 2017/00628; A61B 2017/00592; A61B 2017/00623; A61B 2017/00867

(57) ABSTRACT

The inventive device may include a delivery catheter that remains coupled to an expandable stent portion having an hourglass or “diabolo” shape. The temporary stent device is configured to lodge the shunt portion securely in the atrial septum, preferably the fossa ovalis, to function as an interatrial shunt, allowing blood flow between the left atrium to the right atrium responsive to a pressure differential across the atrial septum. Upon completion of the treatment, a delivery sheath may be used in conjunction with cinching cord coupled to the stent portion to retrieve and remove the temporary shunt device from the patient.

### CROSS-REFERENCE TO RELATED APPLICATIONS

This application claims the benefit of U.S. Provisional Patent Application No. 63/197,279, filed Jun. 4, 2021, the entire contents of which are incorporated by reference herein.

### FIELD OF USE

The present disclosure is directed to systems and methods for creating an interatrial shunt to redistribute blood from one cardiac chamber to another to address pathologies such as heart failure (“HF”), myocardial infarction (“MI”) and pulmonary arterial hypertension (“PAH”).

### BACKGROUND

Pulmonary arterial hypertension (PAH) occurs when the pressure within the blood vessels and lungs becomes too high. PAH may be caused by obstruction in the arteries in the lung such as the development of scar tissue in the blood vessels of the lungs, but in many cases, the cause is unknown. Under normal conditions, the pressure within the right side of the heart and the blood vessels of the lungs is lower than the rest of the body which maximizes oxygenation of the blood in the lungs. With PAH, the heart must work harder under greater pressure to pump blood through the arteries in the lungs, weakening the heart muscles over time. As a result, the heart may be unable to sufficiently pump blood to the lungs to be oxygenated to keep the body functioning normally.

Heart failure is the physiological state in which cardiac output is insufficient to meet the needs of the body or to do so only at a higher filling pressure. There are many underlying causes of HF, including myocardial infarction, coronary artery disease, valvular disease, hypertension, and myocarditis. Chronic heart failure is associated with neurohormonal activation and alterations in autonomic control. Although these compensatory neurohormonal mechanisms provide valuable support for the heart under normal physiological circumstances, they also play a fundamental role in the development and subsequent progression of HF.

For example, one of the body's main compensatory mechanisms for reduced blood flow in HF is to increase the amount of salt and water retained by the kidneys. Retaining salt and water, instead of excreting it via urine, increases the volume of blood in the bloodstream and helps to maintain blood pressure. However, the larger volumes of blood also cause the heart muscle, particularly the ventricles, to become enlarged. As the heart chambers become enlarged, the wall thickness decreases and the heart's contractions weaken, causing a downward spiral in cardiac function. Another compensatory mechanism is vasoconstriction of the arterial system, which raises the blood pressure to help maintain adequate perfusion, thus increasing the load that the heart must pump against.

In low ejection fraction (EF) heart failure, high pressures in the heart result from the body's attempt to maintain the high pressures needed for adequate peripheral perfusion. However, as the heart weakens as a result of such high pressures, the disorder becomes exacerbated. Pressure in the left atrium may exceed 25 mmHg, at which stage fluids from the blood flowing through the pulmonary circulatory system transudate or flow out of the pulmonary capillaries into the pulmonary interstitial spaces and into the alveoli, causing lung congestion and, if untreated, the syndrome of acute pulmonary edema and death.

Table 1 lists typical ranges of right atrial pressure (RAP), right ventricular pressure (RVP), left atrial pressure (LAP), left ventricular pressure (LVP), cardiac output (CO), and stroke volume (SV) for a normal heart and for a heart suffering from HF. In a normal heart beating at around 70 beats/minute, the stroke volume needed to maintain normal cardiac output is about 60 to 100 milliliters. When the preload, after-load, and contractility of the heart are normal, the pressures required to achieve normal cardiac output are listed in Table 1. In a heart suffering from HF, the hemodynamic parameters change (as shown in Table 1) to maintain peripheral perfusion.

TABLE 1

| Parameter | Normal Range | HF Range |
|---|---|---|
| RAP (mmHg) | 2-6 | 6-20 |
| RVSP (mmHg) | 15-25 | 20-80 |
| LAP (mmHg) | 6-12 | 15-50 |
| LVEDP (mmHg) | 6-12 | 15-50 |
| CO (liters/minute) | 4-8 | 2-6 |
| SV (milliliters/beat) | 60-100 | 30-80 |

HF is generally classified as either systolic heart failure (SHF) or diastolic heart failure (DHF). In SHF, the pumping action of the heart is reduced or weakened. A common clinical measurement is the ejection fraction, which is a function of the blood ejected out of the left ventricle (stroke volume) divided by the maximum volume in the left ventricle at the end of diastole or relaxation phase. A normal ejection fraction is greater than 50%. Systolic heart failure generally causes a decreased ejection fraction of less than 40%. Such patients have heart failure with reduced ejection fraction (HFrEF). A patient with HFrEF may usually have a larger left ventricle because of a phenomenon called “cardiac remodeling” that occurs secondary to the higher ventricular pressures.

In DHF, the heart generally contracts normally, with a normal ejection fraction, but is stiffer, or less compliant, than a healthy heart would be when relaxing and filling with blood. Such patients are said to have heart failure with preserved ejection fraction (HFpEF). This stiffness may impede blood from filling the heart and produce backup into the lungs, which may result in pulmonary venous hypertension and lung edema. HFpEF is more common in patients older than 75 years, especially in women with high blood pressure.

Both variants of HF have been treated using pharmacological approaches, which typically involve the use of vasodilators for reducing the workload of the heart by reducing systemic vascular resistance, as well as diuretics, which inhibit fluid accumulation and edema formation, and reduce cardiac filling pressure. No pharmacological therapies have been shown to improve morbidity or mortality in HFpEF whereas several classes of drugs have made an important impact on the management of patients with HFrEF, including renin-angiotensin antagonists, beta blockers, and mineralocorticoid antagonists. Nonetheless, in general, HF remains a progressive disease and most patients have deteriorating cardiac function and symptoms over time. In the U.S., there are over 1 million hospitalizations annually for acutely worsening HF and mortality is higher than for most forms of cancer.

In more severe cases of HFrEF, assist devices such as mechanical pumps are used to reduce the load on the heart by performing all or part of the pumping function normally done by the heart. Chronic left ventricular assist devices (LVAD), and cardiac transplantation, often are used as measures of last resort. However, such assist devices typically are intended to improve the pumping capacity of the heart, to increase cardiac output to levels compatible with normal life, and to sustain the patient until a donor heart for transplantation becomes available. Such mechanical devices enable propulsion of significant volumes of blood (liters/min), but are limited by a need for a power supply, relatively large pumps, and pose a risk of hemolysis, thrombus formation, and infection. Temporary assist devices, intra-aortic balloons, and pacing devices have also been used.

Various devices have been developed using stents to modify blood pressure and flow within a given vessel, or between chambers of the heart. Implantable interatrial shunt devices have been successfully used in patients with severe symptomatic heart failure. By diverting or shunting blood from the left atrium (LA) to the right atrium (RA), the pressure in the LA is lowered or prevented from elevating as high as it would otherwise (left atrial decompression). Such an accomplishment would be expected to prevent, relieve, or limit the symptoms, signs, and syndromes associated of pulmonary congestion. These include severe shortness of breath, pulmonary edema, hypoxia, the need for acute hospitalization, mechanical ventilation, and death.

U.S. Pat. No. 9,067,050 to Gallagher describes an arteriovenous stent assembly including a stent and a pull wire operated flow control mechanism. The stent has a tubular body that defines a fluid passageway between a first end and a second end thereof. The pull wire mechanism includes a portion disposed around the tubular stent in at least one loop. The at least one loop may be selectively tightened or loosened remotely from the stent to regulate the rate of blood flow through the tubular stent.

U.S. Pat. Pub. No. 2013/0178784 to McNamara describes devices and methods for treating heart disease by normalizing elevated blood pressure in the left and right atria of a heart of a mammal. Devices may include an adjustable hydraulic diameter stent portion which can be manually adjusted in vivo. Methods are provided for adjusting the flow rate of the devices in vivo.

Temporary interatrial shunt devices such as those described in U.S. Pat. Pub. No. 2018/0280667 to Keren, the entire contents of which is incorporated herein by reference, include components for retrieving the shunt upon completion of the treatment.

U.S. Pat. Pub. No. 2017/0128705 to Forcucci describes a retrieval device for treating heart failure. Specifically, the retrieval device requires both a first retrieval portion joining the proximal end of a proximal portion of the retrieval device and a second retrieval portion joining the distal end of a distal portion of the retrieval device.

U.S. Pat. No. 5,035,706 to Giantureo describes a self-expanding stent formed of stainless steel wire arranged in a closed zig-zag configuration. The stent is compressible into a reduced diameter size for insertion into and removal from a body passageway. The stent can include a monofilament thread passing through successive eyes at one end of the stent, the thread passing through each eye at least once and through some of the eyes a second time. The trailing ends of the thread extend from the stent and outside the body passageway. The stent can be retrieved from the body passageway by threading a tube over the free ends of the thread until the tube is adjacent the stent. The diameter at one end of the stent is reduced by pulling the free ends of the thread through the tube. A sheath concentrically disposed over the tube is introduced into the body passageway and over the remaining length of the stent to further compress the stent for removal from the passageway.

In view of the foregoing drawbacks of previously known systems and methods, there exists a need for improved in vivo adjustment and retrieval of an interatrial shunt device, particularly a stent having flared ends.

### SUMMARY

The present disclosure overcomes the drawbacks of previously-known systems and methods by providing a retrievable apparatus for temporary, continuously adjustable, shunting of blood across an atrial septum of a patient. For example, the apparatus may include a catheter having a proximal end and a distal end, and a plurality of wires extending distally from the distal end of the catheter and forming a stent having a flared proximal region, a flared distal region, and a neck region therebetween. The stent may transition between a contracted delivery state within a transseptal delivery sheath and an expanded deployed state upon retraction of the delivery sheath, such that the neck region may be positioned within a puncture of the atrial septum of the patient in the expanded deployed state. The apparatus further may include a cinching tube extending distally from the distal end of the catheter toward the neck region of the stent, the cinching tube having a lumen extending therethrough, and a cinching cord having first and second ends, the cinching cord extending around the neck region of the stent such that the first and second ends pass through an outlet of the cinching tube and extend proximally through the lumen of the cinching tube, forming a loop around the neck region. Moreover, movement of the first and second ends of the cinching cord relative to the cinching tube changes the tension in the cinching cord that is looped around the neck region of the stent. For example, moving the first and second ends of the cinching cord proximally relative to the cinching tube causes the loop around the neck region of the stent to tighten, thereby causing the neck region of the stent to transition from the expanded deployed state to a more contracted state. Conversely, relaxing the tension by moving the first and second ends of the cinching cord distally relative to the cinching tube causes the loop around the neck region of the stent to loosen, thereby allowing the superelastic neck region of the stent to expand from a contracted state to a more expanded state. In this way, the orifice through the neck region of the stent may be adjusted to allow greater or lesser flow while the stent is in its deployed configuration within the body of a patient.

The apparatus further may include a sheath having a proximal end, a distal end, and a lumen extending therethrough, the lumen sized and shaped to receive the catheter and the stent in its contracted delivery state. For example, when the stent is disposed within the lumen of the sheath in the contracted delivery state, proximal movement of the sheath relative to the catheter causes the stent to be exposed from the distal end of the sheath and transition from the contracted delivery state to the expanded deployed state. Moreover, the catheter may be slidably disposed within the lumen of the sheath such that as the sheath moves distally relative to the catheter and over a suitably-shaped plurality of wires extending from the distal end of the catheter, the flared proximal region of the stent transitions from the expanded deployed state to the contracted delivery state as the distal end of the sheath slides over it. Further, as the sheath moves distally relative to the catheter from the neck region of the stent toward the flared distal region, the flared distal region of the stent transitions from the expanded deployed state to the contracted delivery state such that the entire stent may be drawn into the sheath.

A diameter of the flared proximal region of the stent may increase from the neck region towards the catheter until reaching an apex of the flared proximal region in the expanded deployed state, and then decrease from the apex of the flared proximal region toward the distal end of the catheter in the expanded deployed state. In addition, the apparatus may include a biocompatible material encapsulating the distal region, the neck region, and at least a portion of the flared proximal region of the stent. For example, the biocompatible material may extend a preselected distance beyond the flared distal region of the stent to thereby reduce injury to surrounding tissue during deployment and retrieval of the stent. Moreover, the biocompatible material may encapsulate the portion of the flared proximal region of the stent between the neck region and the apex of the flared proximal region.

In one embodiment, the cinching tube may extend distally from the distal end of the catheter toward the neck region of stent along an inner surface of the flared proximal region and the neck region of the stent. Alternatively, the cinching tube may extend distally from the distal end of the catheter toward the neck region of stent along an outer surface of the flared proximal region and the neck region of the stent. The neck region of the stent may include a plurality of eyelets disposed circumferentially around the neck region of the stent. Accordingly, the cinching cord may extend through one or more eyelets of the plurality of eyelets around the neck region. Moreover, the biocompatible material may include one or more openings adjacent to the neck region of the stent, the one or more openings aligned with the plurality of eyelets. In some embodiments, one or more eyelets of the plurality of eyelets may include a radiopaque material. Additionally, the cinching tube may include a fairlead adjacent to the outlet of the cinching tube. The fairlead may guide the first and second ends of the cinching cord through the outlet of the cinching tube.

In addition, the catheter further may include a guidewire lumen extending therethrough, the guidewire lumen sized and shaped to receive a guidewire. In another embodiment, the catheter may include a guidewire tube slidably disposed within a guidewire lumen extending therethrough, sized and shaped to receive a guidewire. The guidewire tube may be longer than the catheter, such that it may be extended distally through the neck and beyond the distal flange of the stent. In a preferred embodiment, the guidewire tube may be retracted such that its distal end is even with the distal end of the catheter.

In accordance with another aspect of the present disclosure, a method for temporarily shunting blood across an atrial septum of a patient is provided. For example, the method may include: delivering the distal end of the sheath over a guidewire across the puncture of the atrial septum of the patient into the left atrium; inserting the catheter-stent system over the proximal end of the guidewire and into the proximal hub of the sheath; advancing the catheter until the flared distal region of the stent exits from the distal tip of the sheath, allowing the flared distal region to transition from the contracted delivery state to the expanded deployed state within the left atrium; retracting the sheath and the catheter-stent system as a unit until the flared distal region of the stent encounters the septal wall between the left and right atria; further retracting the sheath proximally while holding the catheter stationary to expose the flared proximal region of the stent, allowing it to transition from the contracted delivery state to the expanded deployed state within the right atrium such that the neck region of the stent is positioned within the puncture of the atrial septum, the flared proximal region of the stent coupled to the distal end of the catheter via a plurality of wires; and shunting blood across the atrial septum via the stent between the atria responsive to a pressure differential across the atrial septum. Further, the method may include adjusting the shunting of blood by moving the first and second ends of the cinching cord that extends around the neck region of the stent relative to the cinching tube to allow the neck to expand to a larger diameter or to cinch the neck to a smaller diameter.

Accordingly, distal movement of the first and second ends of the cinching cord that extends around the neck region of the stent relative to the cinching tube allows the neck region of the stent to transition between a contracted state and an expanded state. Conversely, proximal movement of the first and second ends of the cinching cord relative to the cinching tube tightens the cinching cord around the neck region of the stent, causing it to compress to a smaller diameter.

The method further may include advancing the sheath distally relative to the catheter and over the plurality of wires to transition the flared proximal region of the stent from the expanded deployed state to the contracted delivery state; moving the first and second ends of the cinching cord proximally relative to the cinching tube to transition the neck region of the stent from the expanded deployed state to the contracted delivery state; advancing the sheath distally relative to the catheter over the neck region; further advancing the sheath distally over the flared distal region of the stent to transition the flared distal region of the stent from the expanded deployed state to the contracted delivery state; and removing the sheath, the catheter, and the stent as a unit from the patient.

### BRIEF DESCRIPTION OF THE DRAWINGS

FIGS. 1A and 1B illustrate an exemplary system for temporary shunting between heart chambers constructed in accordance with the principles of the present disclosure.

FIGS. 2A to 2H illustrate an exemplary temporary shunt device constructed in accordance with the principles of the present disclosure.

FIG. 3 shows the temporary shunt device positioned within a puncture of the atrial septum of the patient.

FIG. 4 illustrates an exemplary handle constructed in accordance with the principles of the present disclosure.

FIG. 5 illustrates an exemplary stent cartridge constructed in accordance with the principles of the present disclosure.

FIGS. 6A to 6C illustrate exemplary steps of crimping the stent portion of the temporary shunt device within the stent cartridge of FIG. 5 in accordance with the principles of the present disclosure.

FIGS. 7A to 7G illustrate exemplary steps of loading the temporary shunt device within a delivery sheath over a guidewire in preparation for delivery of the temporary shunt device to the heart of a patient in accordance with the principles of the present disclosure.

FIGS. 8A to 8D illustrate exemplary steps of deploying the temporary shunt device in accordance with the principles of the present disclosure.

FIGS. 9A and 9B illustrate exemplary method steps of adjusting the orifice diameter of an exemplary temporary shunt device in accordance with the principles of the present disclosure.

FIGS. 10A to 10C illustrate another exemplary temporary shunt device constructed in accordance with the principles of the present disclosure.

FIGS. 11A to 11G illustrate exemplary method steps of retrieving the temporary shunt device into the delivery sheath for removal from the patient in accordance with the principles of the present disclosure.

### DETAILED DESCRIPTION

Embodiments of the present invention are directed to devices for temporarily shunting blood between heart chambers, e.g., across the atrial septum of the heart, and thus may be useful in treating subjects suffering from heart failure, pulmonary hypertension, or other disorders associated with elevated left or right atrial pressure. For example, the inventive device may include a delivery catheter that remains coupled to an expandable stent portion via a plurality of wires, the expandable stent having an hourglass or “diabolo” shape, preferably formed of a shape memory metal as described in U.S. Pat. No. 9,629,715 to Nitzan, assigned to the assignee of the present invention, the entire contents of which are incorporated herein by reference. The temporary shunt devices described herein are configured to lodge the stent portion securely in the atrial septum, preferably the fossa ovalis, to function as an interatrial shunt, allowing blood flow between the atria in response to a blood pressure gradient. In one application, the inventive device may be placed to provide short-term acute relief of excess pressure in one of the atria. Upon completion of the treatment, a cinching cord coupled to the stent portion may be used in conjunction with a delivery sheath to retrieve and remove the temporary shunt device from the patient. For example, the cinching cord may be retracted to reduce the orifice of the stent portion, and as the delivery sheath is advanced over the plurality of wires toward an apex of the wires proximal to the proximal flange of the stent portion, the proximal flange gradually compresses to facilitate retrieval of the stent portion into the delivery sheath. In another application, the inventive device may be placed temporarily in the heart of a patient and the shunt diameter adjusted, as described further herein, to determine the optimum degree of shunting to be provided by a subsequent permanent shunt device. Upon completion of this determination, the present temporary adjustable shunt device would be removed from the patient using the delivery sheath and cinching cord as described further herein. In another application, the inventive device may be used in a patient in conjunction with an extracorporeal membrane oxygenation (ECMO) system.

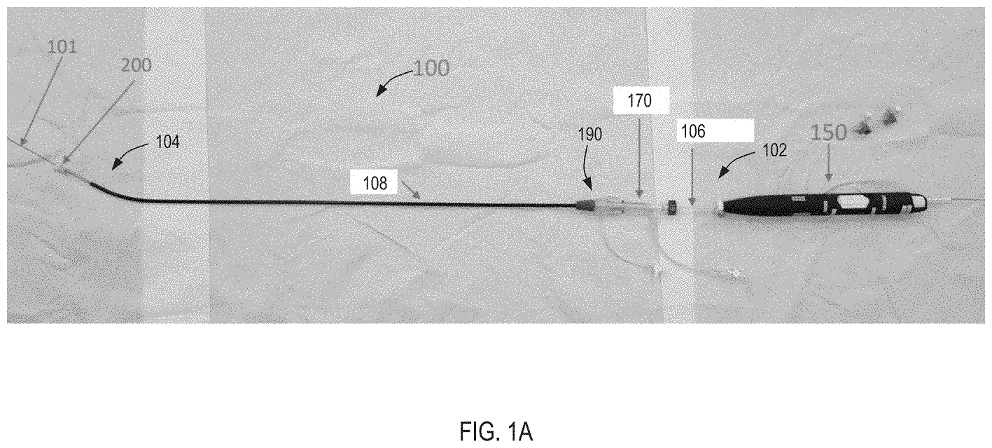

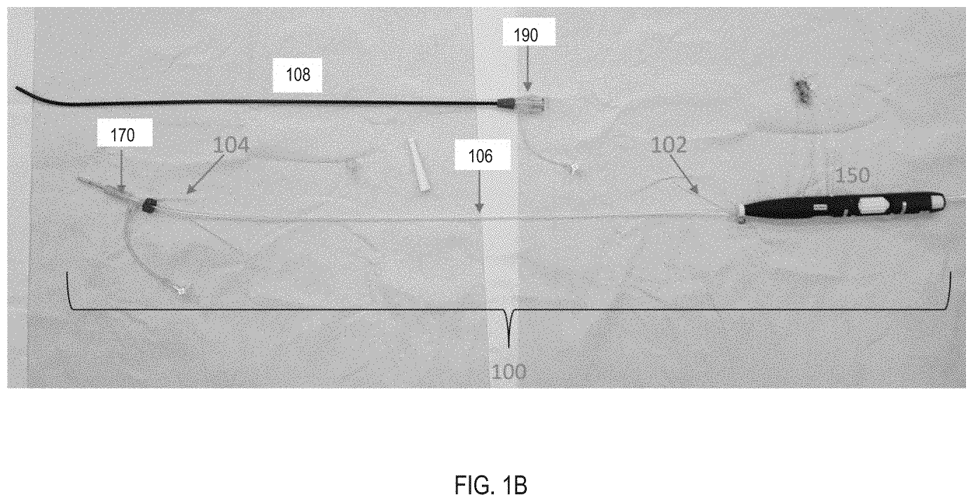

Referring now to FIGS. 1A and 1B, exemplary system 100 for temporary shunting between heart chambers, e.g., the right and left atria, is described. System 100 includes a temporary shunt device having stent portion 200 coupled to distal region 104 of a delivery catheter 106, and handle 150 coupled to proximal region 102 of catheter 106. In addition, system 100 may include delivery sheath 108 having hub 190 containing a hemostatic valve at its proximal end and a lumen sized and shaped to receive delivery catheter 106 and stent portion 200 therethrough, e.g., in a collapsed delivery state. System 100 also may include cartridge 170 slidably disposed on delivery catheter 106 and configured to facilitate insertion of stent portion 200 and delivery catheter 106 into sheath 108 through hub 190. Further, stent cartridge 170 may facilitate the adjustment of the length of system 100, as described in further detail below.

Stent portion 200 along with distal region 104 of delivery catheter 106 and the distal end of sheath 108 are configured to be transvascularly delivered to the patient's atrial septum. The proximal end of delivery catheter 106 may be operatively coupled to handle 150 at proximal region 102, such that each component may be individually actuated via one or more actuators, e.g., buttons, knobs, etc. of handle 150, as described in further detail below. As described in further detail below, system 100 further may include delivery, adjustment, and retrieval elements, e.g., a cinching cord, to facilitate in adjusting stent portion 200 to control blood flow therethrough and for retrieval thereof, such that the proximal ends of the cinching cord also may be operatively coupled to and actuated by handle 150.

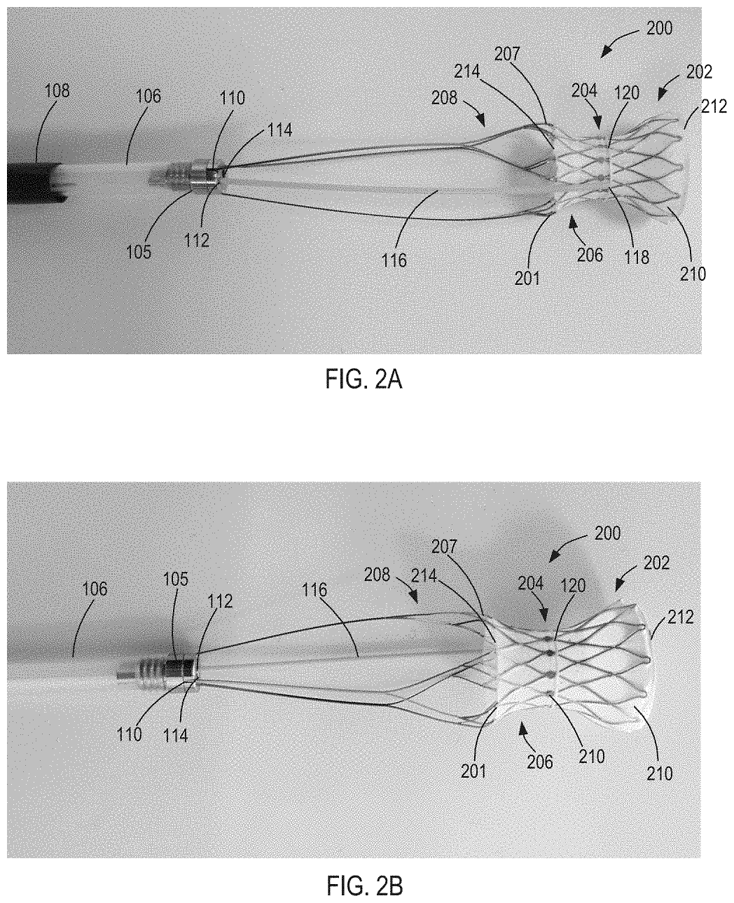

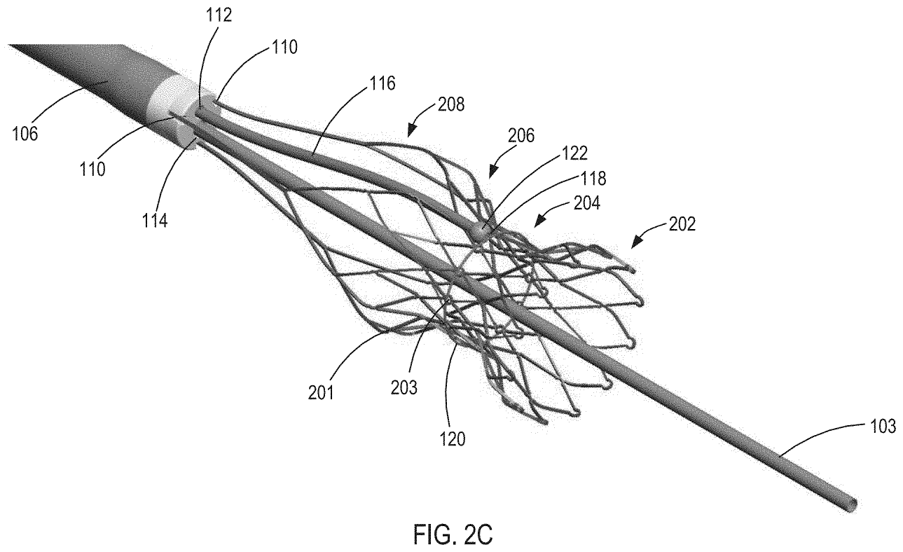

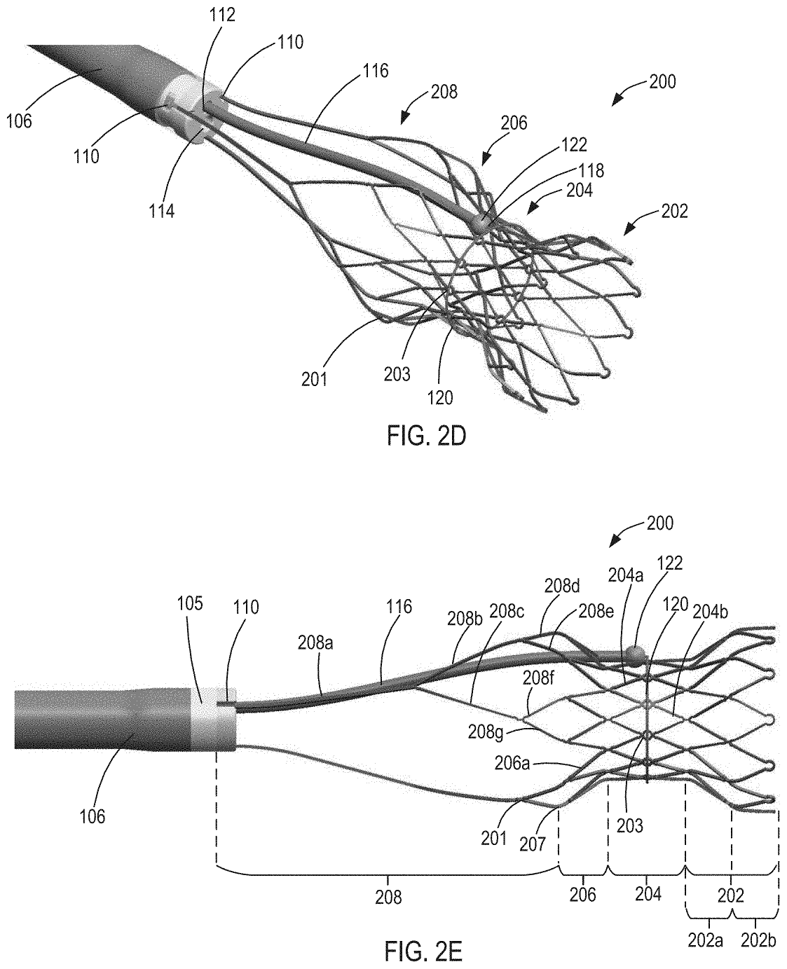

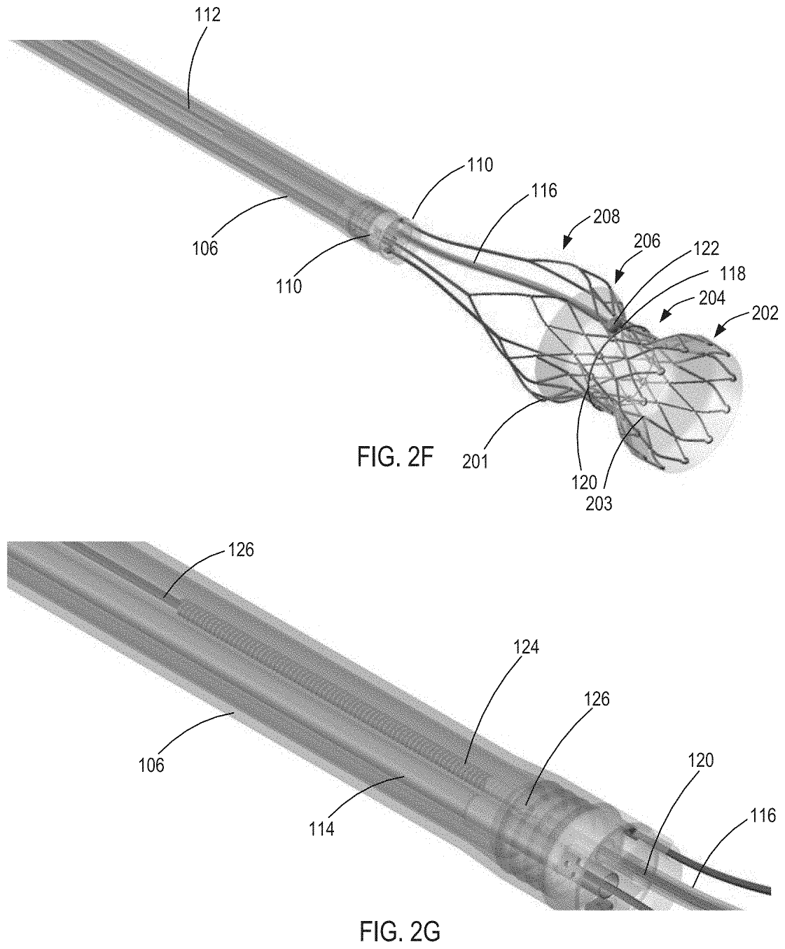

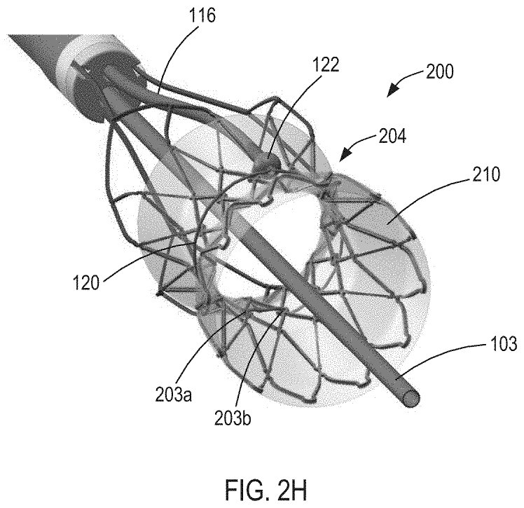

Referring now to FIGS. 2A to 2E, stent portion 200 of system 100 is described. Stent portion 200 may be formed of a plurality of wires extending distally from one or more receptacles 110 of head portion 105 at the distal end of catheter 106. As shown in FIG. 2A, head portion 105 may have three receptacles 110, such that three wires extend distally from the distal end of catheter 106. The plurality of wires extending distally from the distal end of catheter 106 may form expandable stent portion 200, which may have an hourglass or “diabolo” shape in its expanded state, as shown in FIG. 2A. For example, stent portion 200 may include flared distal region 202, flared proximal region 206, neck region 204 position between flared distal region 202 and flared proximal region 206, and proximal connection region 208 that extends from receptacles 110 to the proximal end of flared proximal region 206. Accordingly, stent portion 200 may have, in its expanded state, a cross-sectional area that decreases from the distal end of flared distal region 202 toward neck region 204, increases from neck region 204 toward the proximal end of flared proximal region 206, e.g., at apex 207 between the proximal end of flared proximal region 206 and the distal end of proximal connection region 208, and decreases from apex 207 toward the distal end of catheter 106. As shown in FIG. 2A, each of the plurality of wires extending from receptacles 110 may diverge into a multiple wires, which may each diverge into additional multiple wires to thereby form lattice frame 201 of stent portion 200, as described in further detail below. Lattice frame 201 may be formed from a common metal frame, as illustrated. For example, flared distal region 202, neck region 204, flared proximal region 206, and proximal connection region 208 may be integrally formed from a common frame (e.g., a metal wire frame).

As shown in FIGS. 2A and 2B, at least a portion of stent portion 200 may be encapsulated with biocompatible material 210 to define a continuous shunt that channels blood flow through the passageway of stent portion 200. Biocompatible material 210 may include ultra-high-molecular-weight-polyethylene (UHMWPE), expanded-polytetrafluoroethylene (ePTFE), polyurethane, DACRON (polyethylene terephthalate), silicone, polycarbonate urethane, or pericardial tissue from an equine, bovine, or porcine source, or any combination thereof. For example, flared distal region 202, neck region 204, and at least a portion of flared proximal region 206, but not proximal connection region 208, may be encapsulated with biocompatible material 210. Accordingly, biocompatible material 210 over frame 201 forms inlet/outlet 212 at the distal end of flared distal region 202, and inlet/outlet 214 adjacent to apex 207 between the proximal end of flared proximal region 206 and the distal end of proximal connection region 208, depending on the direction of blood flow across stent portion 200 responsive to the pressure differential across the atrial septum.

Stent portion 200 may be encapsulated with biocompatible material 210 along the outer surface of frame 201 in the direction from the distal end of flared distal region 202 towards the proximal end of flared proximal region 206, and along the inner surface of frame 201 in the direction from the proximal end of flared proximal region 206 towards the distal end of flared distal region 202. In one embodiment, biocompatible material 210 may extend a preselected distance, e.g., 1-2 mm, beyond flared distal region 202 to thereby reduce injury to surrounding tissue during deployment and retrieval of stent portion 200. In addition, system 100 further may include cinching tube 116 extending distally from the distal end of catheter 106 toward neck region 204 of stent portion 200 for housing cinching cord 120, as described in further detail below.

FIGS. 2C to 2E illustrate stent portion 200 with biocompatible material 210 omitted for clarity. Catheter 106 may include guidewire lumen 114 extending through catheter 106 along the longitudinal axis of catheter 106, guidewire lumen 114 sized and shaped to receive guidewire 101 therethrough. In a preferred embodiment, guidewire lumen 114 may further include guidewire insertion and reintroduction tube 103, slidably deposed within guidewire lumen 114 and extending proximally through catheter 106 to its proximal terminus at handle 150. Prior to crimping stent portion 200 into its compressed delivery configuration, guidewire tube 103 may be extended through stent portion 200 as shown in FIG. 2C and stent portion 200 may be crimped onto tube 103, providing a lumen for subsequent passage of guidewire 101 through the crimped stent. Tube 103 permits back-loading of guidewire 101 through the crimped stent and into guidewire lumen 114, as there may otherwise not be any free space in the middle of stent portion 200 when it is crimped. Guidewire tube 103 may be sufficiently rigid such that its lumen does not collapse when stent portion 200 is in its collapsed, crimped delivery state. Once guidewire 101 is inserted through stent portion 200, the distal tip of guidewire tube 103 may be retracted into guidewire lumen 114, for example by actuating a control on handle 150. Further, guidewire tube 103 may be re-extended through stent portion 200 after deployment in the heart to allow reinserting a guidewire into the LA, for example if a guidewire exchange was desired. Extending guidewire tube 103 through stent portion 200 prevents guidewire 101 from passing through connecting wires 208 into the RA instead of through the shunt lumen into the LA, a particular problem with “pigtail” guidewires often used in transseptal procedures. In some embodiments, guidewire lumen 114 may extend along the central axis of catheter 106. Alternatively, guidewire lumen 114 may be offset from the central axis of catheter 106, as shown in FIG. 2C.

FIGS. 2D and 2E illustrate stent portion 200 with guidewire loading tube 103 omitted for clarity. The proximal end of each of the plurality of wires forming frame 201 may include, e.g., a T-shape sized and shaped to engage with receptacles 110 of head portion 105. As shown in FIGS. 2D and 2E, system 100 further may include a cinching tube 116 extending distally from the distal end of catheter 106 toward neck region 204 of stent portion 200. Accordingly, catheter 106 further may include cinching tube lumen 112 extending through at least a portion of catheter 106, cinching tube lumen 112 sized and shaped to receive cinching tube 116 therethrough. For example, in some embodiments, cinching tube 116 may extend through the entire length of catheter 106 along the longitudinal axis of catheter 106. Preferably, cinching tube lumen 112 is offset from the central axis of catheter 106, as shown in FIGS. 2D and 2E. Alternatively, cinching tube lumen 112 may extend along the central axis of catheter 106.

As shown in FIGS. 2F and 2G, the proximal end of cinching tube 116 may be received within cinching tube lumen 112 only at the distal end of catheter 106, thereby providing a passageway between cinching tube lumen 112 and the lumen of cinching tube 116. The lumen of cinching tube 116 is sized and shaped to receive two ends of cinching cord 120 through outlet 118 of cinching tube 116. Cinching cord 120 may be a single wire that extends from outside of the patient, through cinching tube lumen 112 of catheter 106 and cinching tube 116, out of outlet 118 of cinching tube 116, circumferentially around neck region 108, e.g., though eyelets 203 of stent portion 200, and back through outlet 118 of cinching tube 116, cinching tube 116, and cinching tube lumen 112 of catheter 106, such that both free ends of cinching cord 120 are external to the patient's body.

As shown in FIG. 2G, the two free ends of cinching cord 120 may be joined together within cinching tube lumen 112 and affixed together to cinching control rod 126 comprising a metal cinching wire disposed within cinching tube 116 and extending proximally from its connection to the two ends of cinching cord 120 through cinching tube 116 to a location outside the body, where cinching control rod 126 may be pushed or pulled relative to cinching tube 116 to affect movement of the ends of cinching cord 120. Accordingly, movement of the ends of cinching cord 120 relative to cinching tube 116 may transition neck region 204 of stent portion 200 between the contracted delivery state and the expanded deployed state, e.g., via loosening or tightening of the loop of cinching cord 120 about neck region 108. For example, the first and second ends of cinching cord 120 may be pulled proximally relative to cinching tube 116, such that the diameter of the portion of cinching cord 120 surrounding neck region 204 decreases, thereby causing neck region 204 to transition from the expanded deployed state toward the contracted delivery state. Additionally, the first and second ends of cinching cord 120 may be pushed distally relative to cinching tube 116, such that the diameter of the portion of cinching cord 120 surrounding neck region 204 increases, thereby permitting neck region 204 to transition from the contracted delivery state toward the expanded deployed state. Preferably, the cinching cord is a single loop of the polymer, e.g., Ultra-high-molecular-weight polyethylene (UHMWPE), and its two ends are affixed to a metal wire cinching control rod within the cinching tube in the distal portion of the delivery catheter.

Alternatively, a first end of cinching cord 120 may be pulled proximally while the second end of cinching cord 120 remains stationary, such that the diameter of the portion of cinching cord 120 surrounding neck region 204 will decrease, thereby causing neck region 204 to transition from the expanded deployed state toward the contracted delivery state. Additionally, if the first end of cinching cord 120 is eased distally while the second end of cinching cord 120 remains stationary, the diameter of the portion of cinching cord 120 surrounding neck region 204 will increase, thereby permitting superelastic neck region 204 to transition from the contracted delivery state toward the expanded deployed state.

The first and second ends of cinching cord 120 may be operatively coupled to an actuator of handle 150, such that the actuator may be actuated to provide the relative movements of the first and second ends of cinching cord 120 described above. Alternatively, the first and second ends of cinching cord 120 may be affixed to the distal end of cinching control rod 126 that is operatively coupled at its proximal end to an actuator of handle 150.

As shown in FIGS. 2C to 2E, the distal end of cinching tube 116 may include fairlead 122 at its distal end, such that outlet 118 extends through fairlead 122 which is sized and shaped to have a greater radius of curvature than the edge of the distal end of cinching tube 116 to prevent the end of the very thin-walled cinching tube 116 from injuring the fossa ovalis while manipulating the stent, and from damaging cinching cord 120 where it makes a sharp bend as it exits from cinching tube 116. Fairlead 122 may have a low-friction surface sized and shaped to guide cinching cord 120 through outlet 118. For example, fairlead 122 may have a doughnut shape or may be an essentially spherical bead.

Moreover, frame 201 of stent portion 200 may include eyelets 203 disposed circumferentially about neck region 204, such that cinching cord 120 may pass through at least some of eyelets 203. For example, as shown in FIG. 2H, cinching cord 120 may exit cinching tube 116 through fairlead 122, traverse along the outer surface of biocompatible material 210 and frame 201 at neck region 204, pass through eyelet 203a, traverse along the inner surface of biocompatible material 210 and frame 201, pass through eyelet 203a, traverse along the outer surface of biocompatible material 210 and frame 201 at neck region 204, and enter cinching tube 116 through fairlead 122.

Accordingly, biocompatible material 210 encapsulating frame 201 may include one or more openings corresponding to eyelets 203, such that cinching cord 120 may pass through eyelets 203 and biocompatible material 210. In one embodiment, cinching tube 116 may extend from the distal end of catheter 106 and along the outer surface of biocompatible material 210 to neck region 204, and cinching cord 120 may pass around neck region 204 through eyelets 203 such that the first and second ends of cinching cord 120 enters outlet 118 adjacent to the outer surface of biocompatible material 210. In another embodiment, cinching tube 116 may extend from the distal end of catheter 106 and along the inner surface of biocompatible material 210 to neck region 204, and cinching cord 120 may pass through biocompatible material 210 around neck region 204 and through eyelets 203 such that the first and second ends of cinching cord 120 enters outlet 118 adjacent to the inner surface of biocompatible material 210. In some embodiments, one or more eyelets of eyelets 203 may include a radiopaque material such as tantalum to allow easier visualization of diameter of neck region 204, e.g., by fluoroscopy, as shown in FIG. 2B. Additionally or alternatively, radiopaque material may be disposed on flared distal region 202 to facilitate deployment thereof.

As shown in FIG. 2E, each of the plurality of wires extending from receptacles 110 may diverge into multiple wires, which may each diverge into additional multiple wires to thereby form lattice frame 201 of stent portion 200. For example, one of the plurality of wires 208a extending distally from receptacle 110 may diverge into two wires 208b and 208c, and wire 208b may diverge into two additional wires 208d and 208e, and wire 208c may diverge into two additional wires 208f and 208g. Wires 208a-208g may form proximal connection region 208 of stent portion 200. Accordingly, the cross-sectional area of stent portion 200 may increase from receptacle 110 toward the distal ends of wires 208d and 208e in the expanded deployed state.

Flared proximal region 206 of stent portion 200 may be formed by wires 206a having a, e.g., zig-zag shape, extending circumferentially around the longitudinal axis of stent portion 200, such that each of the proximal apices of wire 206a is coupled to the distal end of either wire 208d or wire 208e. For example, each of wires 208d may diverge into two additional wires of wire 206a, which are adjacent to the two additional wires diverging from each of wires 208e, and each of wires 208f may diverge into two additional wires of wire 206a, which are adjacent to the two additional wires diverging from each of wires 208g. Moreover, the cross-sectional area of stent portion 200 may decrease from the distal ends of wires 208d and 208e toward neck region 204, thereby forming apex 207, e.g., a maximum cross-sectional, at the junction between proximal connection region 208 and flared proximal region 206 of stent portion 200.

As shown in FIG. 2E, neck region 204 may have a constant cross-sectional area. Alternatively, neck region 204 may have a parabolic or diabolo shape along its longitudinal axis to thereby facilitate centering of the atrial septum at the narrowest portion of neck region 204. As shown in FIG. 2E, neck region 204 may be formed of two wires 204a and 204b, each having a zig-zag shape and extending circumferentially around the longitudinal axis of stent portion 200. Accordingly, the proximal apices of wire 204a may be coupled to the distal apices of wire 206a, and the distal apices of wire 204a may be coupled to the proximal apices of wire 204b, e.g., via eyelets 203.

Moreover, flared distal region 202 may be formed of first distal region 202a, and second distal region 202b distal to first distal region 202a. Each of first distal region 202a and second distal region 202b may be formed of a wire having a zig-zag shape and each extending circumferentially around the longitudinal axis of stent portion 200. The proximal apices of the wire forming first distal region 202a may be coupled to the distal apices of wire 204b of neck region 204. In addition, the distal apices of the wire forming first distal region 202a may be coupled to the proximal apices of the wire forming second distal region 202b. As shown in FIG. 2E, the cross-sectional area of stent portion 200 may increase across first distal region 202a, e.g., from neck region 204 to the distal end of first distal region 202a, at a larger rate than across second distal region 202b, e.g., from the proximal end of second distal region 202b to the distal end of second distal region 202b. Accordingly, flared distal region 202 may have an overall bell-shape, while flared proximal region 206 may have an overall conical shape.

As will be understood by a person having ordinary skill in the art, although FIGS. 2A to 2E illustrate frame 201 being formed of a lattice of wires having a zig-zap shape, other shapes may be used including, e.g., a sinusoidal shape. For example, frame 201 may be formed of plurality of sinusoidal rings interconnected by a plurality of longitudinally extending struts, as described in U.S. Pat. No. 9,629,715 to Nitzan and U.S. Pat. No. 10,076,403 to Eigler, both assigned to the assignee of the present invention, the entire contents of each of which are incorporated herein by reference. The plurality of longitudinally extending struts of frame 201 may extend from the distal end of flared distal region 202 all the way to the proximal end of proximal connection region 208 at receptacle 110. As will be understood by a person having ordinary skill in the art, proximal connection region 208, flared proximal region 206, neck region 204, and flared distal region 202 may be formed as a single unitary component during manufacturing, such as by laser cutting of a single tube, or alternatively, as separate components coupled to one another.

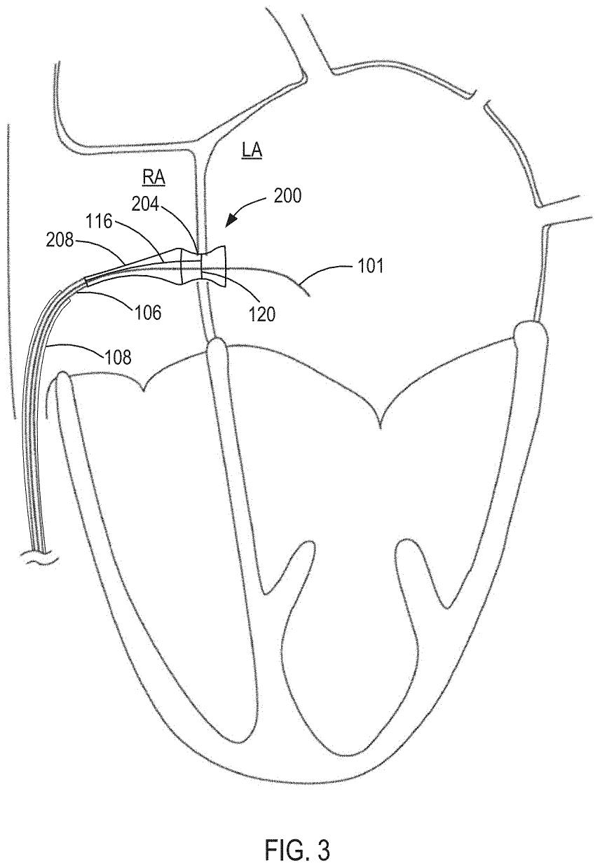

Frame 201 may transition between a contracted delivery state and an expanded deployed state, such that frame 201 is biased toward the expanded deployed state. Accordingly, frame 201 may be advanced in its contracted state through sheath 108 in its contracted delivery state, for delivery to the atrial septum of the patient, e.g., over guidewire 101. Upon exposure from sheath 108, e.g., via retraction of sheath 108 relative to catheter 106, frame 201 may self-expand to transition from the contracted delivery state to the expanded deployed state at the atrial septum as described in further detail below with reference to FIG. 3.

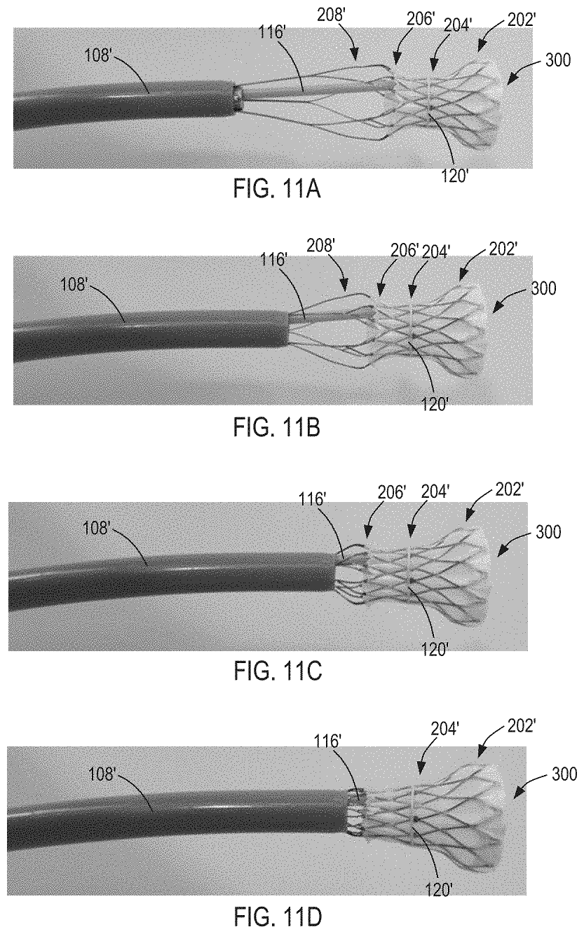

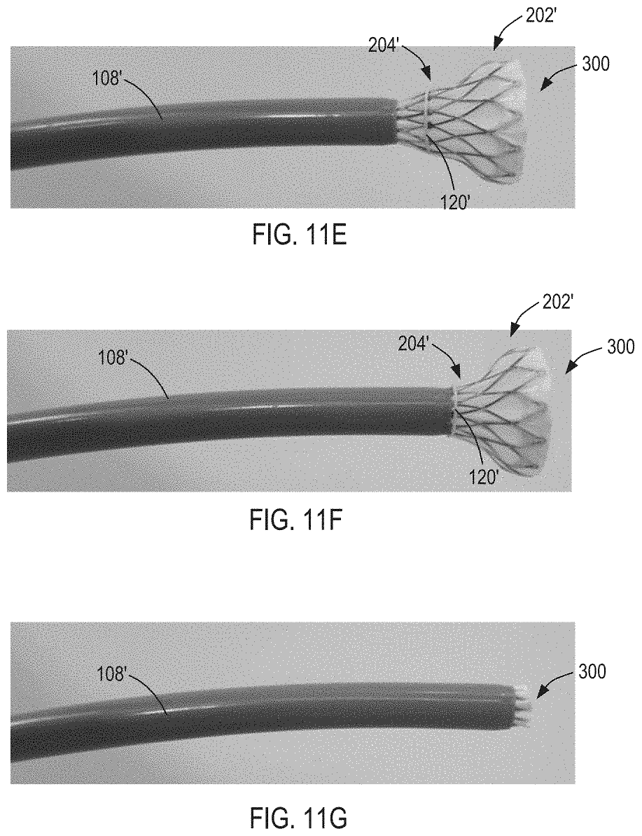

As shown in FIG. 3, the neck region 204 of stent portion 200 may be positioned at the puncture of the atrial septum of the patient, while the flared distal region is deployed within the patient's left atrium LA, the flared proximal region is deployed within the patient's right atrium RA, and the proximal connection region 208 extends from the proximal end of the flared proximal region to the distal end of the catheter 106. Moreover, cinching tube 116 extends from the distal end of the catheter 106 toward the neck region, and cinching cord 120 exits from the outlet of cinching tube 116 and wraps around the neck region. Accordingly, blood may be shunted across the atrial septum via stent portion 200 of system 100 while stent portion 200 is temporarily deployed within the puncture of the atrial septum, e.g., for a few hours, days, or weeks. In some embodiments, system 100 may be used to measure the physiological response to different shunt orifice sizes, e.g., based on the size of the loop of cinching cord 120 while stent portion 200 is temporarily deployed within the puncture of the atrial septum for selecting the optimal size of a treatment device to be implanted at a later time. Upon completion of the treatment or measurements, frame 201 of stent portion 200 may be collapsed into its contracted delivery state within sheath 108 and removed from the patient as described in further detail below with regards to FIG. 11A to 11G.

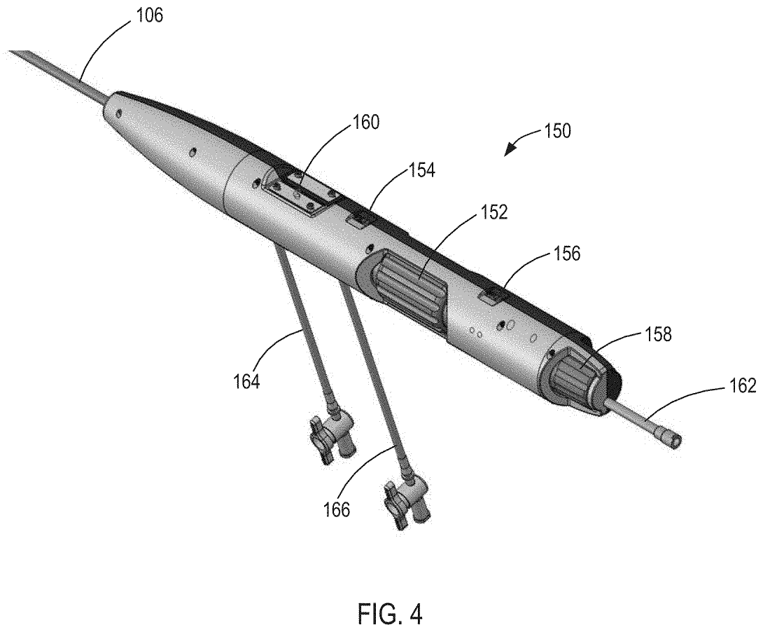

Referring now to FIG. 4, handle 150 is described in further detail. Handle 150 may include knob 152 configured to rotate to extend and/or retract guidewire tube 103 relative to catheter 106. For example, as knob 152 is rotated clockwise, guidewire tube 103 may be retracted proximally relative to catheter 106, and when knob 152 is rotated counter-clockwise, guidewire tube 103 may be extended distally relative to catheter 106, or vice versa. Moreover, handle 150 may include maximum indicator 154 and minimum indicator 156, such that maximum indicator 154 indicates when guidewire tube 103 is fully extended, and minimum indicator 156 indicates when guidewire tube 103 is fully retracted. In addition, handle 150 may include knob 158 configured to rotate to pull cinching control rod 126, and accordingly cinching cord 120, proximally relative to catheter 106 to thereby decrease the diameter of neck region 204, and/or push cinching control rod 126, and accordingly easing cinching cord 120, distally to thereby allowing the diameter of neck region 204 to increase. For example, as knob 158 is rotated clockwise, cinching cord 120 may be pulled proximally relative to catheter 106, and when knob 158 is rotated counter-clockwise, cinching cord 120 may be pushed distally relative to catheter 106, or vice versa. Moreover, handle 150 may include diameter indicator 160, which indicates the current diameter of neck region 204 of stent portion 200, depending on the position of knob 158.

As further shown in FIG. 4, handle 150 may include port 162 extending therethrough having a lumen sized and shaped to receive guidewire 101 therethrough. Accordingly, the lumen of port 162 may be in communication with guidewire tube lumen 114. Moreover, handle 150 may include flushing port 164 for flushing, e.g., cinching tube lumen 112 of catheter 106, and flushing port 166 for flushing, e.g., guidewire tube lumen 114.

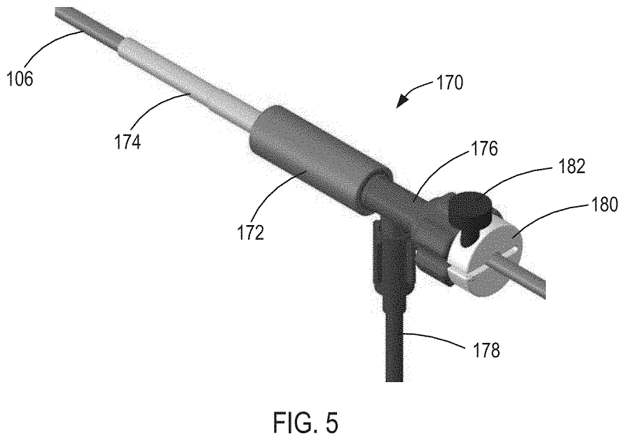

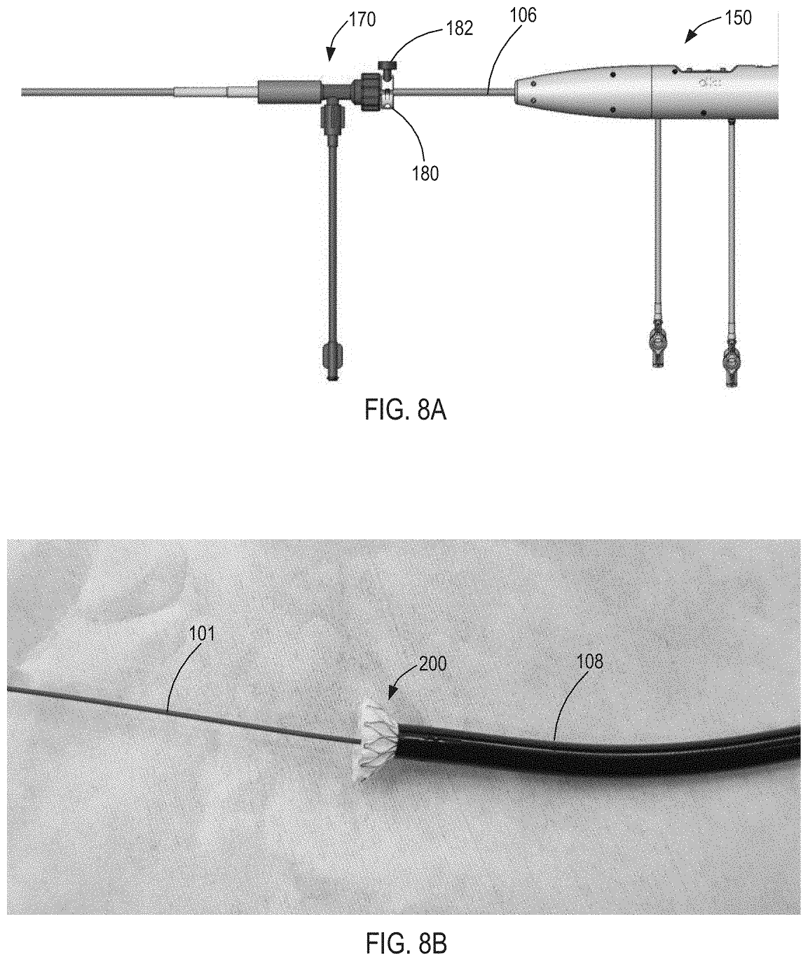

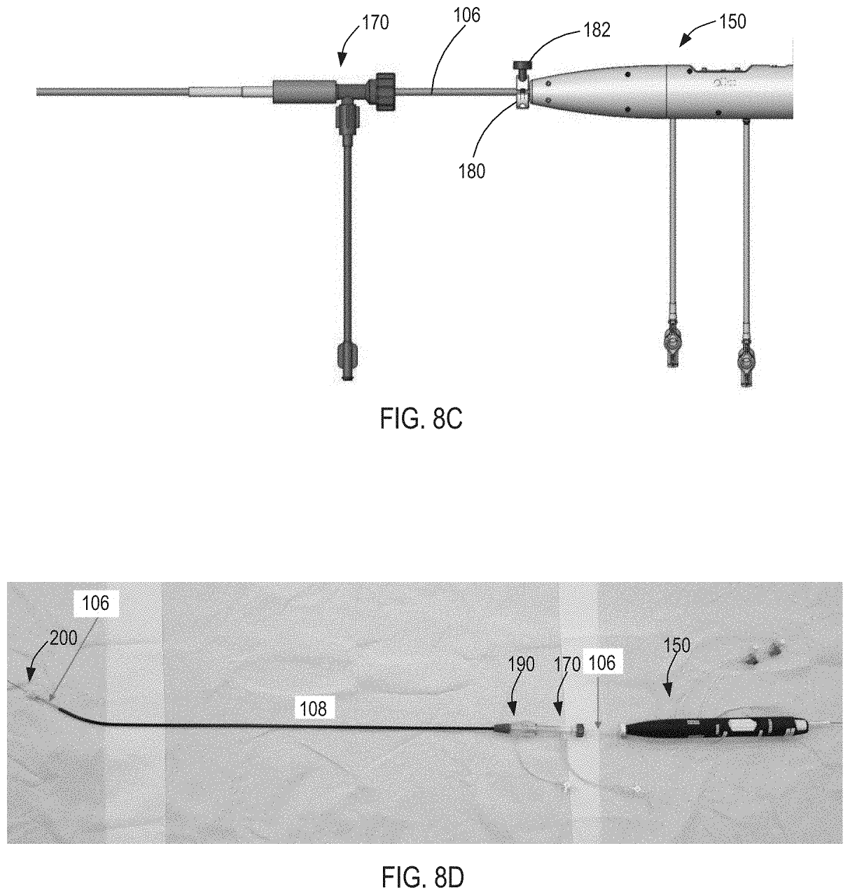

Referring now to FIG. 5, stent cartridge 170 is described in further detail. As shown in FIG. 5, stent cartridge 170 may have a lumen extending therethrough sized and shaped to receive catheter 106 therein. Stent cartridge 170 may have hub stop portion 172 and sheath portion 174 having a smaller outer diameter than hub stop portion 172. The outer diameter of sheath portion 174 may be sized to fit within the lumen of sheath 108 (not shown), such that sheath portion 174 may be inserted within the lumen of sheath 108 to facilitate insertion of stent portion 200, in its collapsed delivery state, into sheath 108, as described in further detail below. Moreover, stent cartridge 170 may include Tuohy-Borst adapter 176, which may be coupled to flushing port 178 for flushing Tuohy-Borst adapter 176. As shown in FIG. 5, system 100 further may include length stopper 180 having knob 182 for adjusting the length of catheter 106, as described in further detail with regard to FIGS. 8A to 8D.

Referring now to FIGS. 6A to 6C, exemplary steps for collapsing stent portion 200 into the lumen of sheath portion 174 of stent cartridge 170 is described. First, as described above with regard to FIG. 4, knob 152 may be rotated until maximum indicator 154 indicates that guidewire tube 103 extends a maximum distance relative to catheter 106. As shown in FIG. 6A, head portion 105 may be visualized through sheath portion 174 to facilitate receipt of stent portion 200 into stent cartridge 170. Next, knob 158 may be rotated to pull cinching cord 120 proximally to decrease the diameter of neck region 204 of stent portion 200 until the diameter of neck region 204 reaches its minimum. As shown in FIG. 6B, stent loader 192 may be used to facilitate collapsing of flared distal region 202 of stent portion 200. For example, stent loader 192 may have a tapered lumen, such that insertion of flared distal region 202 therein causes flared distal region 202 to collapse. When stent portion 200 is in its collapsed delivery state within the lumen of stent loader 192, as shown in FIG. 6B, stent cartridge 170 may be advanced distally toward stent portion 200 until stent portion 200 is received in the lumen of stent cartridge 170 at sheath portion 174, as shown in FIG. 6C. As shown in FIG. 6C, sheath portion 184 may have marker 184 to facilitate retraction of stent portion 200 within sheath portion 174. Sheath portion 174 may then be inserted through sheath hub 190 and within sheath 108, e.g., until the proximal end of sheath 108 engages with hub stop portion 172 of stent cartridge 170. Accordingly, stent portion 200 and catheter 106 may be advanced from stent cartridge 170 through the lumen of sheath 108, and stent cartridge 170 may move proximally relative to catheter 106 until Tuohy-Borst adapter 176 engages with length stopper 180.

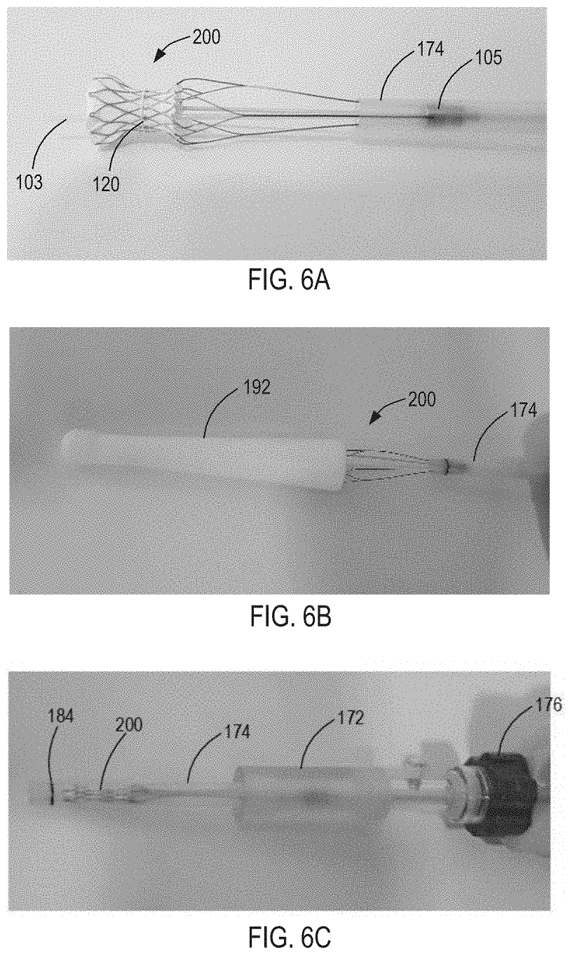

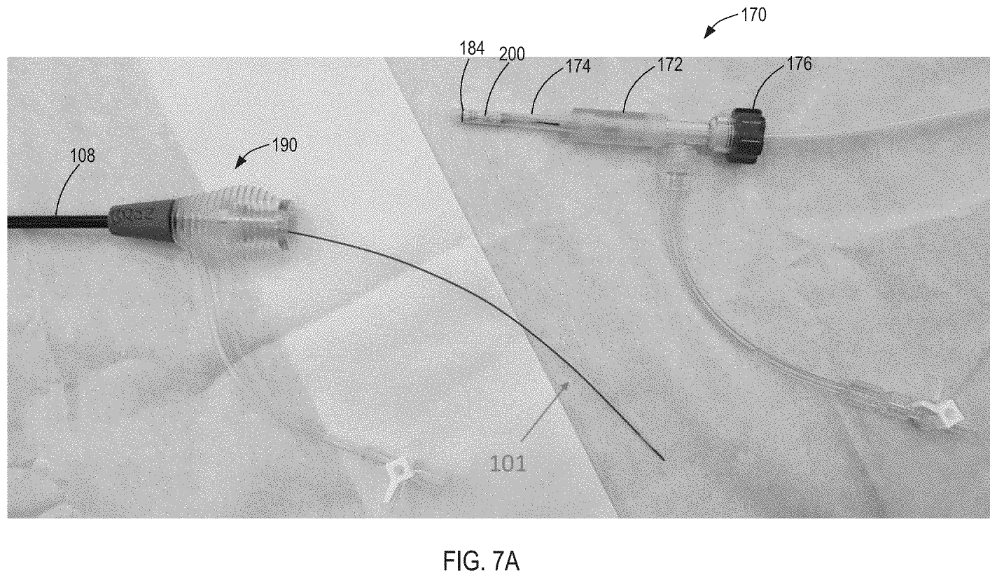

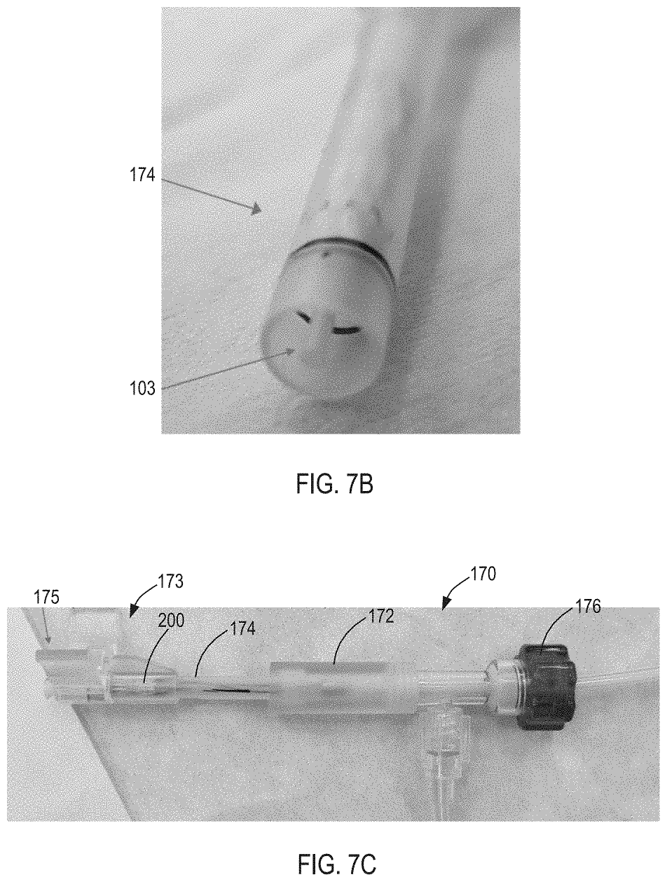

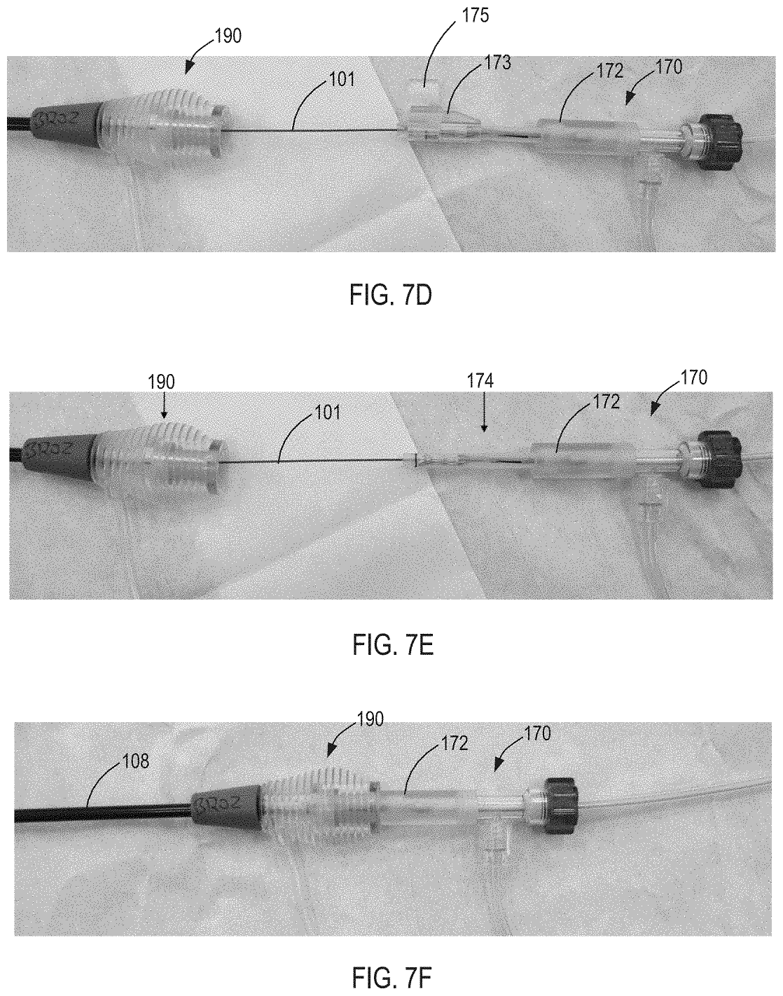

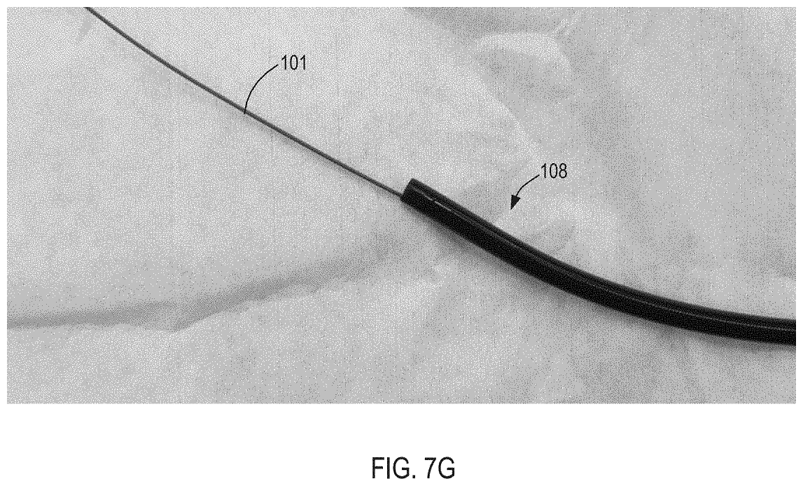

Referring now to FIGS. 7A to 7E, exemplary steps of loading stent portion 200 and the distal end of catheter 106 within delivery sheath 108 over guidewire 101 via stent cartridge 170 and hub 190 of introducer sheath 108 in preparation for delivery of the temporary shunt device to the heart of a patient is provided. As shown in FIG. 7A, guidewire 101 may fed through the lumen of sheath 108 and through the lumen of hub 190, such that the proximal end of guidewire 101 extends proximally from hub 190.

Cartridge 170, stent portion 200 and distal end 104 of delivery catheter 106 may be placed over the proximal end of guidewire 101 by extending the distal end of guidewire tube 103 from its parked location within guidewire lumen 114 to its extended location beyond the distal flange of the crimped stent within cartridge 170, as shown in FIG. 7B, and placing guidewire insertion tool 173 onto the distal end of sheath portion 174 of cartridge 170 as shown in FIG. 7C, such that tool 173 engages the extended guidewire tube 103, ensuring that the proximal end of guidewire 101 slides into guidewire tube 103. Guidewire insertion tool 173 prevents the proximal end of guidewire 101 from accidently passing outside of guidewire tube 103 and damaging the stent's ePTFE encapsulation.

As shown in FIG. 7D, guidewire 101 may be securely inserted into the distal end of guidewire tube 103, and guidewire insertion tool 173 may be removed by sliding it distally to disengage it from guidewire tube 103, then pinching wings 175 of guidewire insertion tool 173 to open its longitudinal slit, allowing it to be removed laterally from guidewire 101, as shown in FIG. 7E. As shown in FIG. 7F, sheath portion 174 of cartridge 170 may then be inserted into sheath hub 190, e.g., until hub stop portion 172 engages with the proximal end of hub 190, allowing delivery system 100 to advance stent portion 200 through the hemostatic valve within hub 105 and into the lumen of sheath 108. Following the insertion of cartridge 170 into sheath hub 190, guidewire tube 103 may be fully retracted into guidewire lumen 114 of catheter 106, e.g., via actuation of knob 152 of handle 150 as described above. Retraction of guidewire tube 103 within guidewire lumen 114 may avoid damaging the LA tissue. FIG. 7G shows guidewire 101 extending from the distal end of sheath 108 when sheath portion 174 of cartridge 170 is inserted into sheath hub 190.

Referring now to FIGS. 8A to 8D, exemplary steps of deploying stent portion 200 from sheath 108 is provided. For example, as shown in FIGS. 8A and 8C, length stopper 180 may be moved proximally or distally relative to catheter 106, and locked in position relative to catheter 106 via knob 182. For example, when length stopper 180 is in the desired position along catheter 106, knob 182 may be actuated to lock length stopper 180 in place. Accordingly, stent cartridge 170 may move proximally relative to catheter 106 until it engages with length stopper 180, thereby adjusting the length of system 100. For half-way deployment of stent portion 200 from sheath 108, stopper 180 may be positioned as shown in FIG. 8A. After verifying the location of the sheath distal tip in the LA, stent portion 200 may be half-deployed by advancing the delivery system until length stopper 180 encounters cartridge 170, as shown in FIG. 8B. For full deployment of stent portion 200 from sheath 108, knob 182 may be actuated to unlock length stopper 180, such that stopper 180 may be moved proximally toward handle 150, as shown in FIG. 8C. Accordingly, cartridge 170, hub 190, and sheath 108 may be moved proximally along catheter 106 to thereby fully expose stent portion 200 from the distal end of sheath 108, as shown in FIG. 8D.

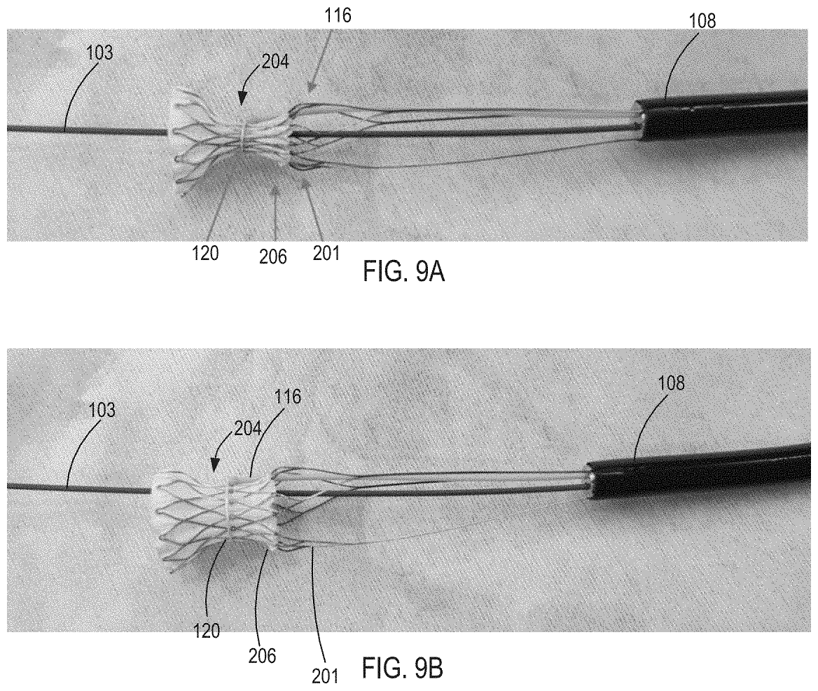

Referring now to FIGS. 9A and 9B, exemplary method steps of adjusting the orifice diameter of stent portion 200 is provided. As described above, the free ends of cinching cord 120 may be pull proximally through cinching tube 116 such that the diameter of the loop of cinching cord 120 around neck region 204 decreases, thereby decreasing the diameter of neck region 204, as shown in FIG. 9A. Reducing the diameter of neck region 204 via cinching cord 120 may also reduce the diameter of proximal region 206, as shown in in FIG. 9A, thereby facilitating re-sheathing of sheath 108 over the wires of frame 201 of stent portion 200. FIG. 9A illustrates stent 200 with a minimum neck region diameter, e.g., maximum crimping via cinching cord 120. Moreover, as shown in FIG. 9B, the tension of cinching cord 120 may be released by pushing the free ends of cinching cord 120 distally relative to cinching tube 116, thereby allowing the diameter of the loop of cinching cord 120 around neck region 204 to increase. Accordingly, the diameter of neck region 204 will also increase.

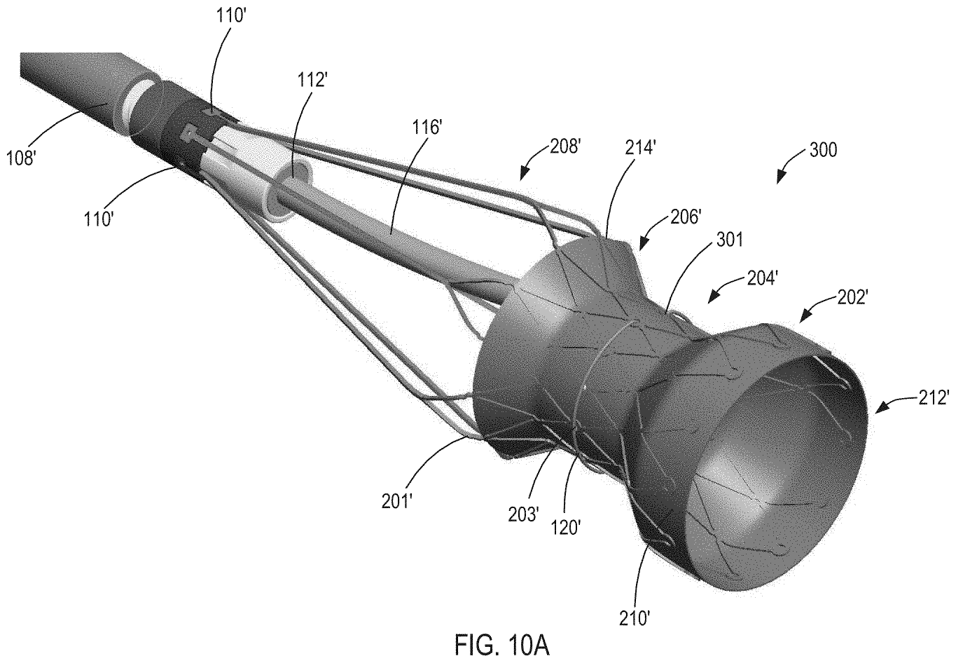

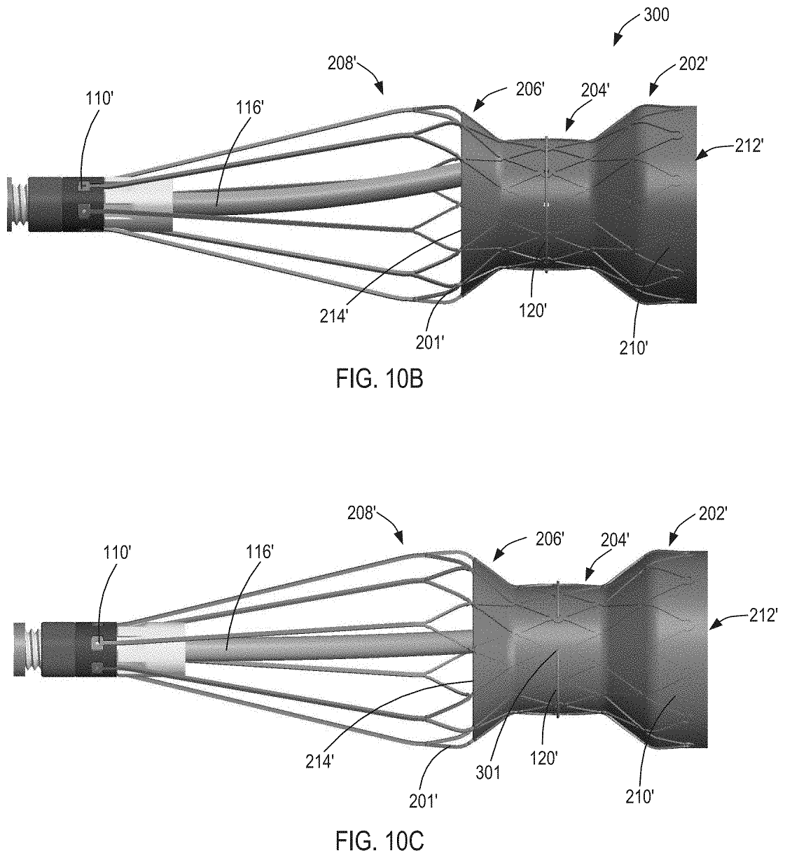

Referring now to FIGS. 10A to 10C, alternative stent portion 300 is provided. Stent portion 300 may be constructed similar to stent portion 200 of FIG. 2A, wherein like components are identified by like-primed reference numbers. For example, catheter 106′ corresponds with catheter 106, receptacles 110′ correspond with receptacles 110, cinching tube lumen 112′ corresponds with cinching tube lumen 112, cinching tube 116′ corresponds with cinching tube 116, proximal connection region 208′ corresponds with proximal connection region 208, flared proximal region 206′ corresponds with flared proximal region 206, neck region 204′ corresponds with neck region 204, flared distal region 202′ corresponds with flared distal region 202, biocompatible material 210′ corresponds with biocompatible material 210, eyelets 203′ correspond with eyelets 203, and cinching cord 120′ corresponds with cinching cord 120. Stent portion 300 differs from stent portion 200 in that stent portion 300 may not include a guidewire lumen extending through catheter 106′. Accordingly, as shown in FIGS. 10A-10C, cinching tube lumen 112′ may be coaxial with catheter 106′, such that cinching tube 116′ extends from the center of the distal end of catheter 106′ toward neck region 204′.

Moreover, as shown in FIGS. 10A-10C, frame 201′ of stent portion 300 may be formed of more than three wires extending from receptacles 110′ of catheter 106′. For example, stent portion 300 may include six wires extending from receptacles 110′ to form the stent. Like frame 201, each of the six wires of frame 201′ may diverge into two additional wires, which may each diverge into two additional wires to thereby form a lattice of frame 201′. As shown in FIGS. 10A-10C, the plurality of wires may only diverge into additional wires once to form proximal connection region 208′, such that the next divergence into additional wires occurs at the apex between proximal connection region 208′ and flared proximal region 206′. As will be understood by a person having ordinary skill in the art, less than three, four, five, or more than six wires may extend from the distal end of the catheter to form the stent portion.

As described above with regard to stent portion 200, although FIGS. 10A-10C illustrate frame 201′ being formed of a lattice of wires having a zig-zap shape, other shapes may be used including, for example, a sinusoidal shape. For example, frame 201′ may be formed of plurality of sinusoidal rings interconnected by a plurality of longitudinally extending struts, as described in U.S. Pat. No. 9,629,715 to Nitzan and U.S. Pat. No. 10,076,403 to Eigler, both assigned to the assignee of the present invention, the entire contents of each of which are incorporated herein by reference. The proximal end of each of the plurality of wires forming frame 201′ may include, e.g., a T-shape sized and shaped to engage with receptacles 110′.

As shown in FIGS. 10A-10C, cinching tube 116′ may extend from the distal end of catheter 106′ along an inner surface of biocompatible material 210′ towards neck region 204′. Accordingly, cinching cord 120′ may extend across biocompatible material 210′, e.g., via eyelets 203′, and enter the outlet of cinching tube 116′ adjacent to the inner surface of biocompatible material 210′. Alternatively, cinching tube 116′ may extend from the distal end of catheter 106′ along an outer surface of biocompatible material 210′ towards neck region 204′. Accordingly, cinching cord 120′ may extend across biocompatible material 210′, e.g., via eyelets 203′, and enter the outlet of cinching tube 116′ adjacent to the outer surface of biocompatible material 210′.

Referring now to FIGS. 11A to 11G, exemplary method steps of retrieval of the temporary shunt device are provided. Although FIGS. 11A to 11G illustrate retrieval of stent portion 300, any of the systems for temporary shunting described herein may be retrieved in a similar manner. As shown in FIG. 11A, in the fully expanded deployed state, sheath 108′ is positioned over catheter 106′, such that the distal end of sheath 108′ is adjacent to the distal end of catheter 106′ and covering at least a portion of receptacle 110′ (not shown). In this configuration, when stent portion 300 is fully deployed at the puncture of the atrial septum of the patient, blood may be shunted across the atrial septum via stent portion 300 responsive to pressure differential across the atrial septum, as shown in FIG. 3.

The initiation of the retrieval of stent portion 300 is illustrated in FIG. 11B, where sheath 108′ is advanced distally relative to catheter 106′ such that the distal end of sheath 108′ slides over the plurality of wires extending from the distal end of catheter 106′, e.g., proximal connection region 208′, and cinching tube 116′. Accordingly, as sheath 108′ is advanced over proximal connection region 208′, the inward radial force applied to the plurality of wires of proximal connection region 208′ causes flared proximal region 206′ to transition from its expanded deployed state to its contracted delivery state. Sheath 108′ is advanced over proximal connection region 208′ until the distal end of sheath 108′ is adjacent to the apex between proximal connection region 208′ and flared proximal region 206′, such that flared proximal region 206′ is in its contracted delivery state, as shown in FIG. 11C.

As shown in FIG. 11D, the free ends of cinching cord 120′ may be moved proximally relative to the proximal end of cinching tube 116′, e.g., either manually or by actuation of handle 150, to thereby reduce the diameter of cinching cord 120′ that is wrapped around neck region 204′, which causes neck region 204′ to transition from its expanded deployed state toward its contracted delivery state. Alternatively, the first end of cinching cord 120′ may be pulled proximally relative to the cinching tube 116′ while the second end of cinching cord 120′ remains stationary, such that the portion of cinching cord 120′ that is wrapped around neck region 204′ is pulled through the outlet of cinching tube 116′, to thereby reduce the diameter of cinching cord 120′ around neck region 204′. As will be understood by a person having ordinary skill in the art, cinching cord 120′ may reduce the diameter of neck region 204′ to a desired size to facilitate retrieval of stent portion 300, e.g., to a partially or fully contracted state.

Next, sheath 108′ may be further advanced distally relative to catheter 106′ such that the distal end of sheath 108′ receives flared proximal region 206′ as shown in FIG. 11E, and neck region 204′, as shown in FIG. 11F. If neck region 204′ is not collapsed to its fully contracted delivery state by actuation of cinching cord 120′, the distal end of sheath 108′ will fully collapse neck region 204′ as sheath 108′ is advanced distally relative to catheter 106′ over neck region 204′, as shown in FIG. 11F. Sheath 108′ may further be advanced distally relative to catheter 106′ such that the distal end of sheath 108′ receives at least a portion of flared distal region 202′, thereby causing flared distal region 202′ to transition from its expanded deployed state to its contracted delivery state, as shown in FIG. 11G. Accordingly, system 100 may be removed from the patient. In a preferred embodiment, stent portion 300 may be pulled entirely within sheath 108′ by moving catheter 106′ proximally relative to sheath 108′ prior to removal from the patient.

The inventors tested a prototype of the described device in a living sheep. After gaining femoral venous access a standard transseptal puncture was performed and an Anchorwire™ transseptal guidewire was placed through the puncture into the LA. Following well-established practice, a 15F Ventura™ introducer sheath was placed over this guidewire through the atrial septum and into the LA.

The stent was loaded into the cartridge using a tapered loader and sliding the cartridge over the proximal connection wires, the distal connection wires, and the proximal, neck and distal portions of the stent, positioning the entire stent within the narrow loading tube portion of the cartridge, all at the distal end of the delivery catheter. The loading tube portion of the cartridge was then inserted into the delivery sheath hub, and the delivery catheter was advanced through the sheath until just the distal flange of the stent emerged from the distal end of the sheath.

The stent and delivery system were further prepared on the bench to set a length stopper on the delivery catheter between the handle and the cartridge to prevent advancing the neck of the stent beyond the end of the sheath. Accurately setting this stopper allows the stent to be confidently half-deployed within the left atrium (i.e. only the distal flange released from the sheath) by advancing the delivery catheter until this stopper encounters the cartridge. Once the stopper has been set in the correct location, the half-deployed stent is retracted back into the sheath as shown in FIGS. 11A-11G by pulling back on the delivery catheter while holding the sheath in place. The delivery catheter is further withdrawn from the sheath until the stent is pulled into the narrow loading tube portion of the cartridge, as shown in FIG. 6C. At this point the cartridge may be decoupled from the sheath and the sheath may be fitted with an appropriate dilator and placed over a guidewire into the patient, across the atrial septum and into the left atrium following standard transseptal technique. The dilator is then withdrawn from the sheath, leaving the sheath and guidewire in place in the LA. The stent portion and the distal end of the catheter may then be inserted through the lumen of the sheath via the sheath hub and cartridge, as described above, such that the stent portion may be deployed across the atrial septum of the patient.

TABLE 2 shows results of an exemplary study of the inventive system in an ovine animal model. After deploying the stent across the atrial septum as described above, the control handle was actuated to select shunt orifice diameter by adjusting the length of cinching cord 120 as described above. Pre-calibrated control handle settings from 5 mm to 8 mm were selected in 1 mm steps, then varied from 8 mm back down to 4 mm. At each setting the shunt orifice diameter was determined by intracardiac ultrasound (ICE) and the stent frame diameter was measured by X-ray using the delivery system head diameter as a reference. Despite some apparent backlash in the X-ray measurements, the data shows that shunt orifice diameter may be increased and decreased over the design range with reasonably accurate control over flow orifice diameter.

TABLE 2

| | Control | Orifice | Frame |
|---|---|---|---|
| | Knob | Diameter (mm) | Diameter (mm) |
| Step | Setting | by ICE | by X-ray |
| 1 | 5 | 5.4 | 5.4 |
| 2 | 6 | 6.5 | 6.7 |
| 3 | 7 | 7.9 | 7.6 |
| 4 | 8 | 9.3 | 8.6 |
| 5 | 7 | 7.2 | 8.6 |
| 6 | 6 | 6.1 | 7.7 |
| 7 | 5 | 5.7 | 7.3 |
| 8 | 4 | 4.4 | 5.5 |

Following the above deployment, adjustment and measurements, the delivery sheath was advanced over the apex region of the connecting wires, compressing the proximal flared region of the stent. The cinching cord was maximally tightened to compress the neck region of the stent, allowing the sheath to be advanced over the wires extending from the delivery system to the apex at the proximal end of the stent, thereby compressing the proximal flared end of the stent to allow it to be pulled into the delivery sheath, following which the remainder of the stent may be easily pulled entirely into the delivery sheath, and the entire system safely removed from the body. It would be obvious to one skilled in the art that the ability to vary the size of an interatrial shunt would advantageously allow a physician to determine the optimum shunt diameter for the treatment of conditions involving excessive pressure in one of the atria, such as heart failure or pulmonary artery hypertension.

While various illustrative embodiments of the invention are described above, it will be apparent to one skilled in the art that various changes and modifications may be made therein without departing from the invention. The appended claims are intended to cover all such changes and modifications that fall within the true scope of the invention.

What is claimed:

1. An apparatus for temporarily shunting blood across an atrial septum of a patient, the apparatus comprising:
a catheter having a proximal region and a distal region;
a plurality of wires extending distally from the distal region of the catheter and forming a stent having a flared proximal region, a flared distal region, and a neck region therebetween, the stent configured to transition between a contracted delivery state and an expanded deployed state, the neck region configured to be positioned within a puncture of the atrial septum of the patient in the expanded deployed state;
a cinching tube extending distally from the distal region of the catheter toward the neck region of the stent, the cinching tube having a lumen extending therethrough; and
a cinching cord having first and second ends, the cinching cord extending around the neck region of the stent such that the first and second ends pass through an outlet of the cinching tube and extend through the lumen of the cinching tube,
wherein movement of the first and second ends of the cinching cord relative to the cinching tube causes the neck region of the stent to transition between the contracted delivery state and the expanded deployed state.

2. The apparatus of claim 1, further comprising a sheath having a proximal end, a distal end, and a lumen extending therethrough, the lumen sized and shaped to receive the catheter and the stent in the contracted delivery state.

3. The apparatus of claim 2, wherein, when the stent is disposed within the lumen of the sheath in the contracted delivery state, proximal movement of the sheath relative to the catheter causes the stent to be exposed from the distal end of the sheath and transition from the contracted delivery state to the expanded deployed state.

4. The apparatus of claim 2, wherein the catheter is slidably disposed within the lumen of the sheath such that as the sheath moves distally relative to the catheter and over the plurality of wires extending from the distal region of the catheter, the flared proximal region of the stent transitions from the expanded deployed state to the contracted delivery state.

5. The apparatus of claim 4, wherein, when the neck region of the stent is in the contracted delivery state, as the sheath moves distally relative to the catheter from the neck region toward the flared distal region of the stent, the flared distal region of the stent transitions from the expanded deployed state to the contracted delivery state.

6. The apparatus of claim 1, wherein a diameter of the flared proximal region of the stent increases from the neck region towards the catheter until an apex of the flared proximal region in the expanded deployed state, and then decreases from the apex of the flared proximal region toward distal region of the catheter in the expanded deployed state.

7. The apparatus of claim 1, further comprising a biocompatible material encapsulating the distal region, the neck region, and at least a portion of the flared proximal region of the stent.

8. The apparatus of claim 7, wherein the biocompatible material extends a preselected distance beyond the flared distal region of the stent to thereby reduce injury to surrounding tissue during deployment and retrieval of the stent.

9. The apparatus of claim 7, wherein a diameter of the flared proximal region of the stent increases from the neck region towards the catheter until an apex of the flared proximal region in the expanded deployed state, and then decreases from the apex of the flared proximal region toward distal region of the catheter in the expanded deployed state, such that the biocompatible material encapsulates the distal region, the neck region, and the portion of the flared proximal region of the stent between the neck region and the apex of the flared proximal region.

10. The apparatus of claim 7, wherein the biocompatible material comprises one or more openings adjacent to the neck region of the stent, the one or more openings aligned with a plurality of eyelets disposed circumferentially around the neck region of the stent, and wherein the cinching cord extends through one or more openings in the biocompatible material and through one or more of the plurality of eyelets around the neck region.

11. The apparatus of claim 1, wherein the cinching tube extends distally from the distal region of the catheter toward the neck region of stent along an inner or outer surface of the flared proximal region and the neck region of the stent.

12. The apparatus of claim 1, wherein the neck region of the stent comprises a plurality of eyelets disposed circumferentially around the neck region of the stent, and wherein the cinching cord extends through one or more eyelets of the plurality of eyelets around the neck region.

13. The apparatus of claim 12, wherein one or more eyelets of the plurality of eyelets comprise a radiopaque material.

14. The apparatus of claim 1, wherein the cinching tube comprises a fairlead adjacent to the outlet of the cinching tube, the fairlead configured to guide the first and second ends of the cinching cord through the outlet of the cinching tube.

15. The apparatus of claim 1, wherein the catheter further comprises a guidewire lumen extending therethrough, the guidewire lumen sized and shaped to receive a guidewire tube slideably disposed therewithin, the guidewire tube lumen sized and shaped to receive a guidewire.

16. A method for temporarily shunting blood across an atrial septum of a patient, the method comprising:
delivering a distal end of a sheath across a puncture of the atrial septum of the patient within a first atrium, the sheath having a catheter slidably disposed therein, the catheter coupled to a stent configured to transition between a contracted delivery state within a lumen of the sheath, and an expanded deployed state;
moving the sheath proximally relative to the catheter to transition a flared distal region of the stent from the contracted delivery state to the expanded deployed state within the first atrium;
further retracting the sheath proximally relative to the catheter to transition a flared proximal region of the stent from the contracted delivery state to the expanded deployed state within a second atrium such that a neck region of the stent between the flared proximal region and the flared distal region of the stent is positioned within the puncture of the atrial septum, the flared proximal region of the stent coupled to a distal region of the catheter via a plurality of wires; and
shunting blood across the atrial septum via the stent between the first atrium and the second atrium responsive to a pressure differential across the atrial septum,
wherein proximal movement of first and second ends of a cinching cord that extends around the neck region of the stent and through a cinching tube extending from the neck region of the stent through the delivery catheter causes a reduction of an orifice diameter of the neck region of the stent.

17. The method of claim 16, wherein distal movement of the first and second ends of the cinching cord causes an increase of the orifice diameter of the neck region of the stent.

18. The method of claim 16, further comprising:
advancing the sheath distally relative to the catheter and over the plurality of wires to transition the flared proximal region of the stent from the expanded deployed state to the contracted delivery state;
moving the first and second ends of the cinching cord proximally relative to the cinching tube to transition the neck region of the stent from the expanded deployed state to the contracted delivery state;
advancing the sheath distally relative to the catheter and over the neck region and at least a portion of the flared distal region of the stent to transition the flared distal region of the stent from the expanded deployed state to the contracted delivery state; and
removing the sheath, the catheter, and the stent from the patient.

19. The method of claim 16, wherein the distal region, the neck region, and at least a portion of the flared proximal region of the stent are encapsulated with a biocompatible material.

20. The method of claim 19, wherein a diameter of the flared proximal region of the stent increases from the neck region towards the catheter until an apex of the flared proximal region, and then decreases from the apex of the flared proximal region toward distal region of the catheter, such that the biocompatible material encapsulates the distal region, the neck region, and the portion of the flared proximal region of the stent between neck region and the apex of the flared proximal region.
